(19) United States
(12) Patent Application Publication
(10) Pub. No.: US 2023/0212621 A1
(43) Pub. Date: Jul. 6, 2023

(54) PROCESS FOR SYNTHESIS OF POLY-GAMMA-GLUTAMIC ACID

(71) Applicant: COUNCIL OF SCIENTIFIC AND INDUSTRIAL RESEARCH, New Delhi (IN)

(72) Inventors: Mahesh Shantappa DHARNE, Pune, Maharashtra (IN); Pranav Girijavallabhan NAIR, Pune, Maharashtra (IN)

(21) Appl. No.: 18/001,117

(22) PCT Filed: Jun. 9, 2021
(86) PCT No.: PCT/IN2021/050558

§ 371 Date: Dec. 8, 2022

(30) Foreign Application Priority Data
Jun. 9, 2020 (IN) 202011024273

(51) Int. Cl. C12P 13/02; C12N 1/20

(52) U.S. Cl. CPC C12P 13/02; C12N 1/205; C12R 2001/07

(57) ABSTRACT

The present invention overcomes the problem of processes yielding poor quantities of poly-gamma-glutamic-acid (γ-PGA) by providing a process and a novel medium for synthesis of γ-PGA in the presence of Bacillus paralicheniformis MCC 196. Wherein, the novel medium comprises tomato waste alone for cost effective and greener production of γ-PGA with yield of 40 g/L. In another process, non-sterile fermentation employs the use of a medium high in sugar content. Wherein, sucrose is used in concentrations up to 50% to obtain more than 280 g/L of poly gamma glutamic acid. The disclosed process is scalable and provides immense economic advantages.

### FIELD OF THE INVENTION

The present invention relates to a cost-effective process for the synthesis of poly-gamma-glutamic acid (γ-PGA) from tomato waste and high concentration of sucrose. More particularly, the present invention relates to a process for the synthesis of highly pure poly-gamma-glutamic acid in high yield and in the presence of Bacillus paralicheniformis H6 (MCC 0196).

### BACKGROUND AND PRIOR ART OF THE INVENTION

Poly-gamma-glutamic acid (γ-PGA) is a polymer of the amino acid glutamic acid (GA). Poly-gamma-glutamic acid (γ-PGA) is the form where the peptide bonds are between the α-amino group of glutamic acid (GA) and the γ-carboxyl group at the end of the GA side chain. γ-PGA is a high value, eco-friendly, biodegradable polymer produced mainly by Bacillus spp. These water-soluble, anionic biopolymers are coupled via amide bonds between the L/D-glutamic acid monomers. Owing to its biodegradable, non-toxic and non-immunogenic properties, it has importance in the biomedical field, cosmetics, food industry, wastewater treatment, and other applications. It has the potential to be used for protein crystallization, as a soft tissue adhesive and a non-viral vector for safe gene delivery.

Recently, there is an increase in the demand for the use of biopolymers worldwide, thus they are on the verge of replacing conventional petro-based polymers. However, the main drawback that prevents wider commercialization of biopolymers is the cost involved in biosynthesis when compared to their conventional counterparts. Out of all the biopolymers known, γ-PGA is one of the most expensive biopolymer.

γ-PGA is a major constituent of the Japanese food natto, which has drawn attention by a boom in health care, and studies on natto mucilage have also progressed. The natto mucilage is mainly constituted of poly-gamma-glutamic acid produced by Bacillus subtilis var. natto. The bacterial synthesis of γ-PGA has been attempted in the art, albeit high production costs involved in preparation of medium and specific constituents.

Perusal of prior literature relating to γ-PGA reveals that there are quite a few bacteria which produce poly-gamma-glutamic acid regardless of addition or no addition of L-glutamic acid including B. subtilis, B. lichniformis and B. megaterium amongst others. An attempt to use agro bio resources such as rice bran for the synthesis of poly gamma glutamic acid has also been made, however through a few batch process.

Even though there are potent γ-PGA producing strains available, the cost of production remains exorbitant. Thus, the only solution to this problem is to reduce the overall cost of production by using wastes that can replace all the nutrients required for γ-PGA production. To achieve this, one should have a profound knowledge of various nutritional factors that affect γ-PGA production. Contrastingly, as there is enormous potential in India as well as in European countries for the valorization of surplus waste, it may be possible to direct the waste for economic and feasible production of γ-PGA.

The main drawback that limits the commercialization of γ-PGA is its high production cost. Thus, there is an absolute need for an alternate production medium which is economically feasible and can substitute the existing medium thereby decreasing the overall cost of production. Glucose, L-glutamic acid and citric acid are the major nutrients that are required for γ-PGA production. Substituting the conventionally used nutrient sources with agricultural waste will greatly reduce the cost of γ-PGA production and also will result in the valorization of waste.

γ-PGA is a sleeping giant in the field of biopolymers. According to its commercial rate, around 100 mg costs Rs.24000/-. γ-PGA's market value is exorbitant due to the expensive substrate used for its production. Therefore, the conventional petro-based polymers materials, which γ-PGA is actually conceptualized to replace, will not be a workable solution to combat the ecological issues. Therefore, there is an absolute need for a high producer strain as well as a low-cost substrate for production of γ-PGA.

It is also pertinent to note that the various species and strains of Bacillus reported yield poor quantities of poly gamma glutamic acid adding to their other disadvantages. Further the processes that have reported improved yields have employed addition of nutrients, while there are possibilities of exploiting natural waste as potential substrates for providing processes with advantages including substantial improvement in yields as well as accruing economic advantages. Such advantages are not only due to the enhanced output, but also due to the decreased cost of medium and nutrients to be added in the process.

### OBJECTIVE OF THE INVENTION

The objective of the present invention is to provide a process for the synthesis of highly pure poly-gamma glutamic acid (γ-PGA) from inexpensive substrates.

The main objective of the present invention is to provide a process for the synthesis of γ-PGA from inexpensive waste materials.

The important objective of the present invention is to provide a process for the synthesis of γ-PGA from tomato waste and sucrose, which is an inexpensive substrate, and its use will valorize surplus waste into value-added commodity polymers.

Another objective of the present invention is to reduce the production cost of poly-gamma glutamic acid (γ-PGA).

### SUMMARY OF THE INVENTION

Accordingly, to accomplish the objectives of the invention, the present disclosure provides an improved process for the synthesis of poly-gamma-glutamic acid is disclosed herein comprising the steps of:
a) incubating a raw material having ingredients selected from at least 20% w/w of a carbon source, at least 7% w/w of glutamic acid, at least 1% w/w of a nitrogen source, at least 1% w/w of citric acid and 6×10⁸ to 6×10⁹ CFU/ml of Bacillus paralicheniformis MCC 0196 for 12-48 hours at 28-45° C. at 5.0-8.0 pH to obtain a broth with a supernatant and
b) separating the broth and supernatant, adding ice cold methanol to the supernatant to obtain the poly-gamma-glutamic acid of molecular weight in the range of 500-1000 kDa and yield ranging from 40-300 g/lt. In a preferred embodiment, the Bacillus organism is Bacillus paralicheniformis MCC 0196 isolated from honey.

The carbon, nitrogen, glutamic acid and citric acid in the process are selected from a natural source or a synthetic source. Further, the source for carbon is sugars such as but not limited to glucose, fructose, sucrose, trehalose, xylose and lactose. In a preferred embodiment, the source of nitrogen are organic sources selected from peptone, tryptone, yeast extract and beef extract or inorganic sources selected from ammonium sulfate, ammonium chloride, ammonium citrate, ammonium acetate and ammonium nitrate. The process further comprises a source of potassium, magnesium, calcium and iron.

In another embodiment, the natural source of carbon, nitrogen, glutamic acid and citric acid is a tomato waste. Further, the tomato waste can be used alone or in combination with a source of potassium, magnesium, calcium and iron.

In a more preferred embodiment, the synthesis of poly-gamma-glutamic acid comprises:
a) incubating a raw material having ingredients selected from at least 20% w/w of a carbon source, at least 7% w/w of glutamic acid, at least 1% w/w of a nitrogen source, at least 1% w/w of citric acid, NH₄Cl 6 g/lt; K₂HPO₄ 1 g/lt; MgSO₄, ·7H₂O 0.5 g/lt; CaCl₂·2H₂O 0.2 g/lt; FeCl₃·7H₂O 0.03 g/lt and 6×10⁸ CFU/ml of Bacillus paralicheniformis MCC 0196 for 36 hours at 28° C. at 7.5 pH to obtain a broth with a supernatant and
b) centrifuging the broth at 10000 rpm for 30 minutes to obtain a clear supernatant and adding ice cold methanol to precipitate the poly-gamma-glutamic acid.

In an aspect, the present disclosure provides a culture medium comprising tomato waste, said tomato waste comprising at least 50 mg/ml glucose, 50 mg/l fructose, 10 mg/ml glutamic acid and 5 mg/ml citric acid is disclosed. The culture medium comprising tomato waste for the synthesis of poly-gamma glutamic acid is prepared by a process comprising:
a) grinding a tomato waste prepared from over-ripened and/or waste tomatoes;
b) centrifuging the ground tomato waste of step (a) to obtain a debris free supernatant; and
c) concentrating said supernatant and adjusting the pH to 7.5 to obtain said culture medium.

### BRIEF DESCRIPTION OF THE DRAWINGS

FIG. 1(a) depicts the growth of H6 isolate on nutrient agar plate, and the FIG. 1(b) depicts the molecular phylogenetic analysis by neighbor joining method;

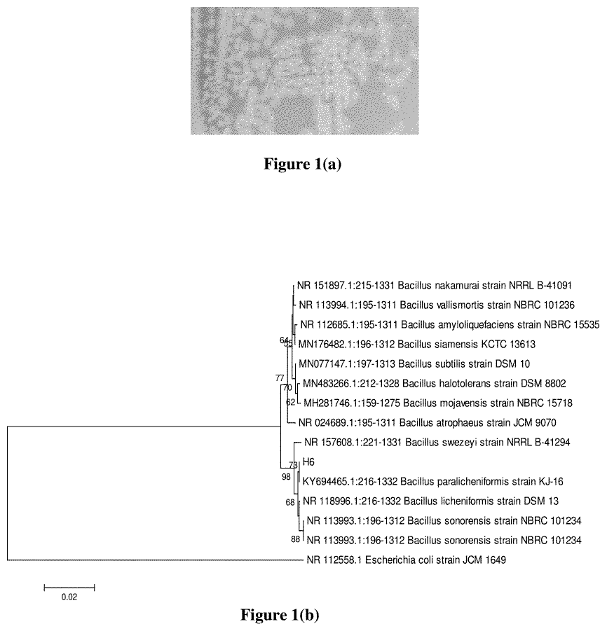

FIG. 2(a) depicts the production of γ-PGA production from synthetic medium, and the FIG. 2(b) depicts the effect of time on γ-PGA from synthetic medium;

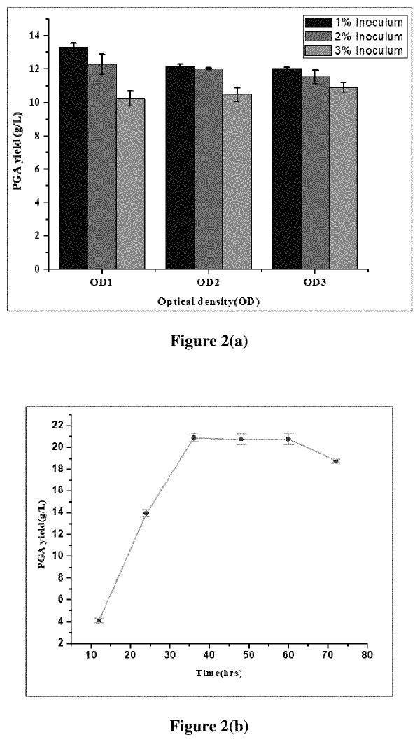

FIG. 3(a) depicts the effect of temperature on γ-PGA production from synthetic medium, and the FIG. 3(b) depicts the effect of pH on γ-PGA production from synthetic medium;

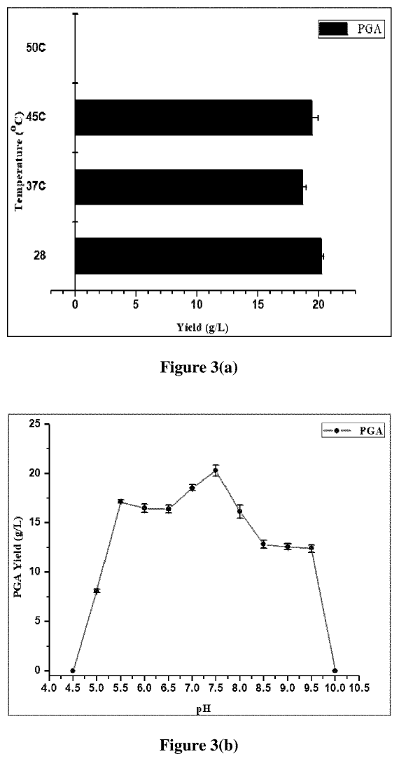

FIG. 4(a) depicts the effect of carbon sources on γ-PGA production, and the FIG. 4(b) depicts the effect of nitrogen sources on γ-PGA production;

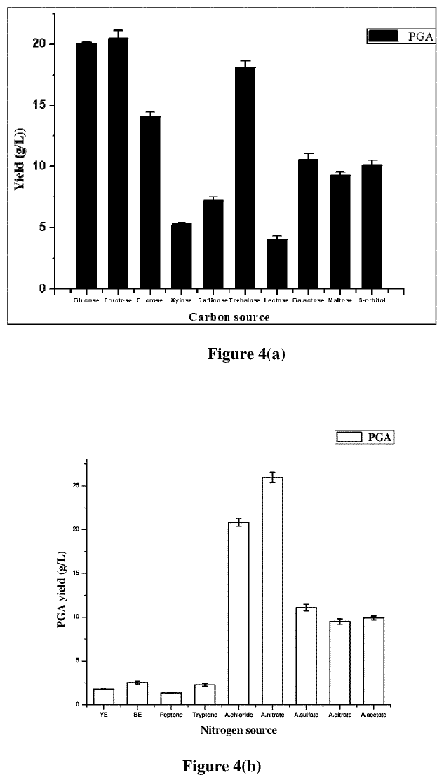

FIG. 5(a) depicts γ-PGA production using tomato waste medium containing ammonium nitrate and trace element, and the FIG. 5(b) depicts γ-PGA production using tomato waste as a substitute for synthetic production medium;

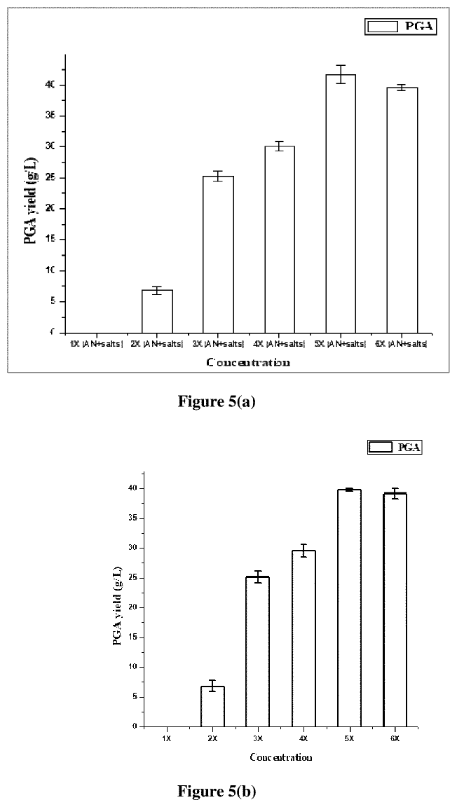

FIG. 6 depicts the comparative study on γ-PGA production using tomato waste and synthetic medium (B medium);

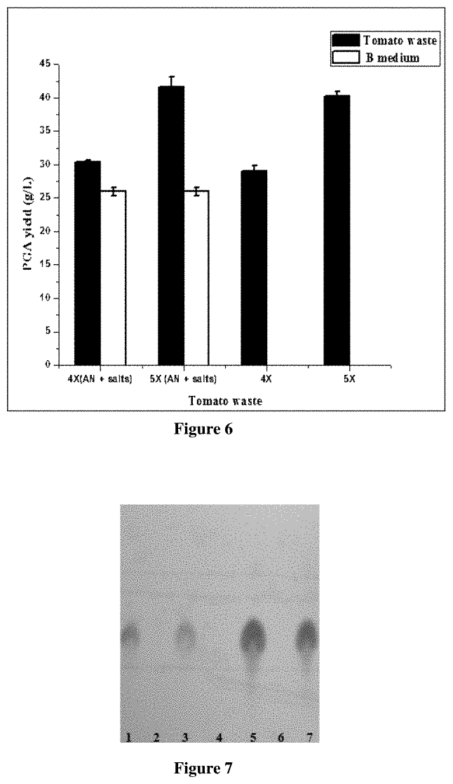

FIG. 7 depicts the TLC of biopolymer; 1) Standard L-glutamic acid, 2) Std γ-PGA (Sigma) unhydrolyzed, 3) Std γ-PGA (Sigma) hydrolyzed, 4) Biopolymer from synthetic medium unhydrolyzed, 5) Biopolymer from synthetic medium hydrolyzed, 6) Biopolymer from Tomato waste medium unhydrolyzed, 7) Biopolymer from Tomato waste medium hydrolyzed;

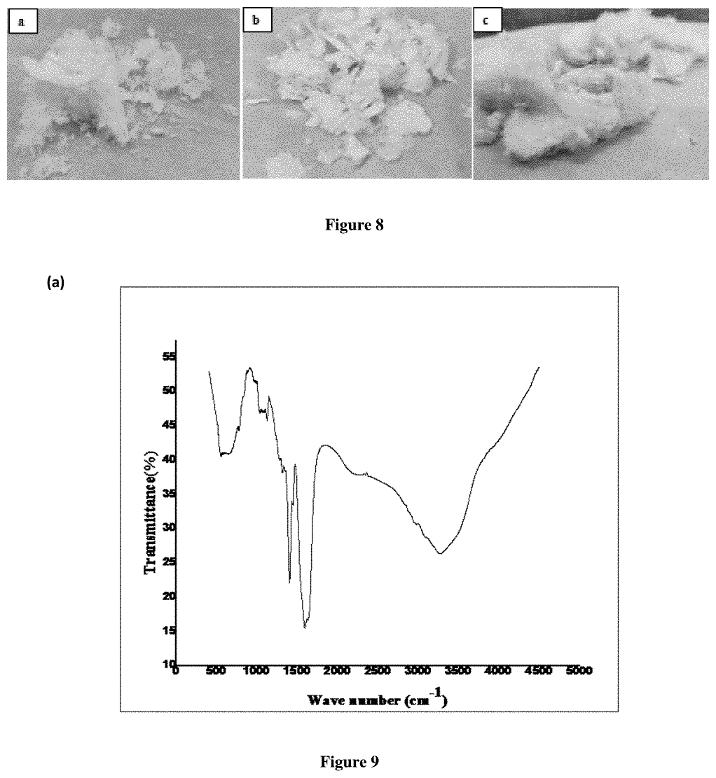

FIG. 8(a) depicts the standard γ-PGA obtained from Sigma, and FIG. 8(b) depicts the biopolymer from synthetic production medium, and FIG. 8(c) depicts the biopolymer from tomato waste medium;

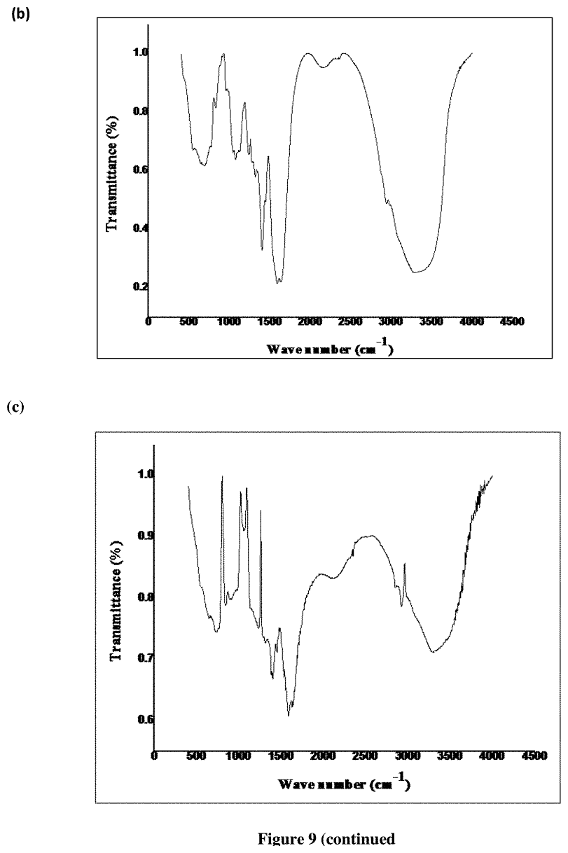

FIG. 9 depicts the FTIR analysis of (a) standard γ-PGA, (b) biopolymer from tomato waste medium and (c) biopolymer from synthetic production medium;

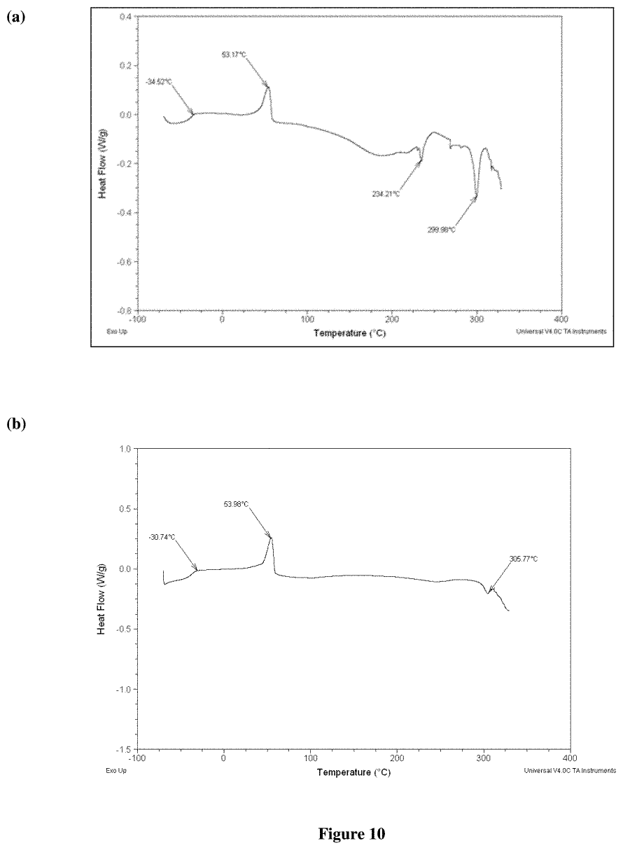

FIG. 10 depicts the DSC analysis of (a) Standard γ-PGA, (b) biopolymer from tomato waste and (c) biopolymer from synthetic medium;

FIG. 11 depicts the NMR analysis of (a) Standard γ-PGA, (b) biopolymer from tomato waste, (c) biopolymer from synthetic medium;

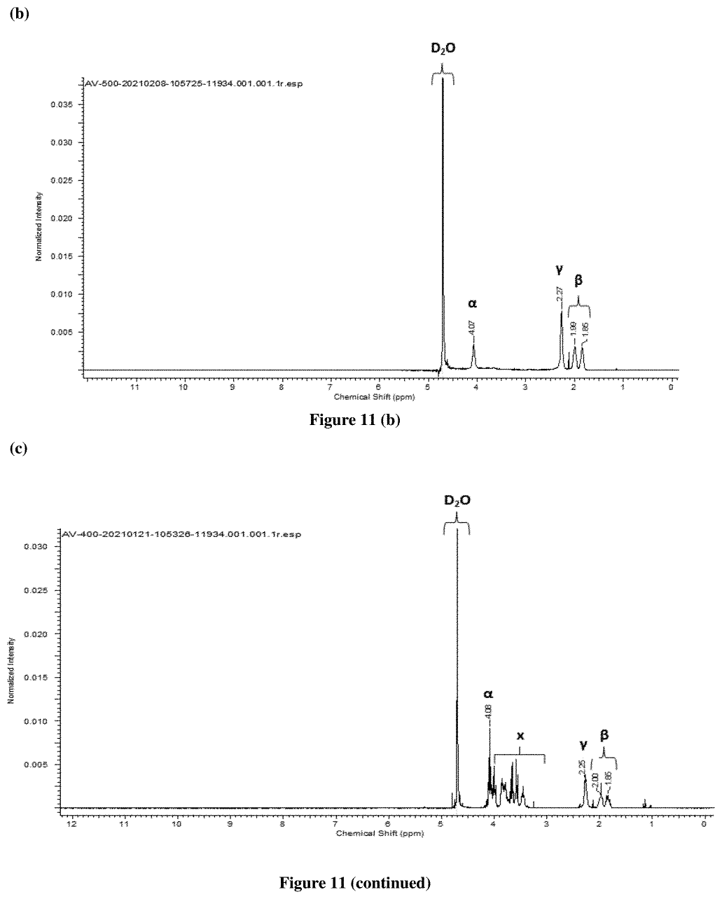

FIG. 12 depicts the HPLC analysis of sugars (a) Standard fructose, (b) Standard glucose, (c) Standard fructose+glucose, (d) Tomato waste, (e) Tomato waste 5× concentrated;

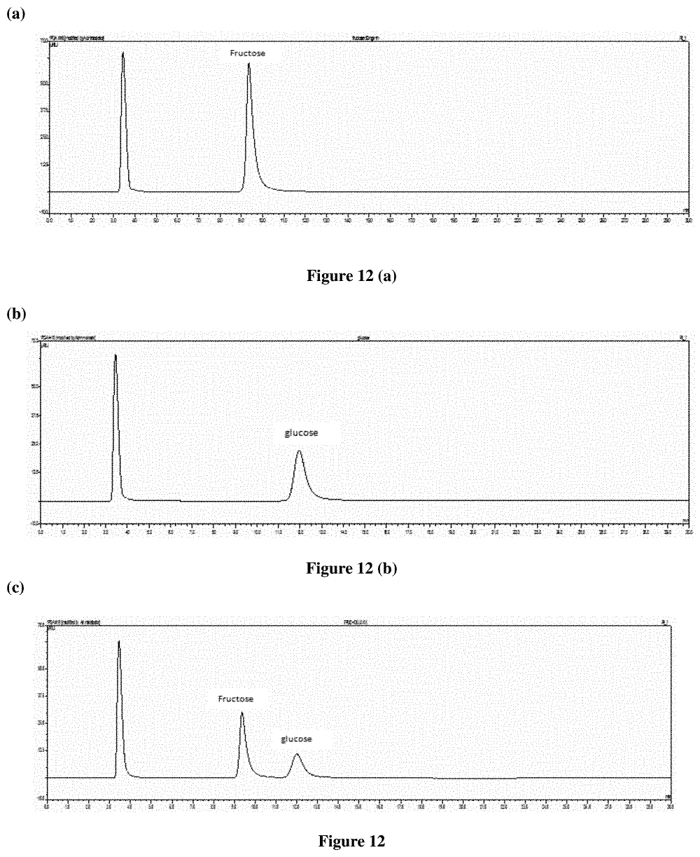

FIG. 13 depicts the HPLC analysis of L-glutamic acid (a) Standard L-glutamic acid, (b) Tomato waste, (c) Tomato waste 5× concentrated;

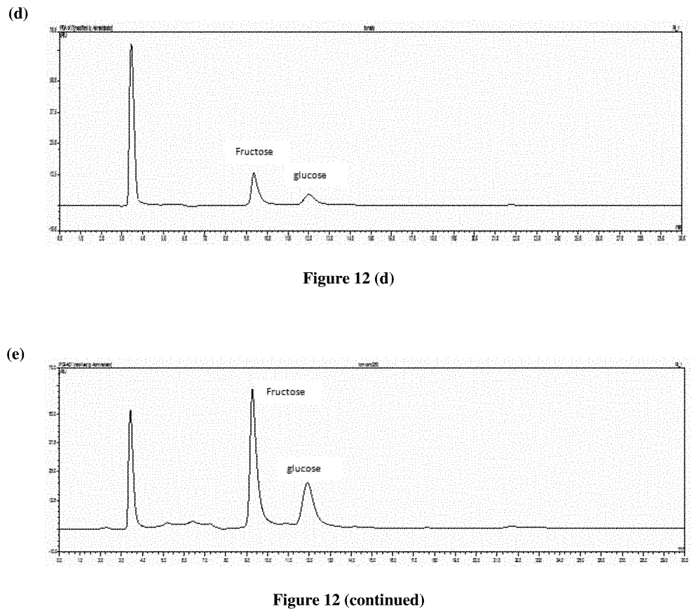

FIG. 14 depicts the HPLC analysis of citric acid (a) Standard citric acid, (b) Tomato waste, (c) Tomato waste 5× concentrated;

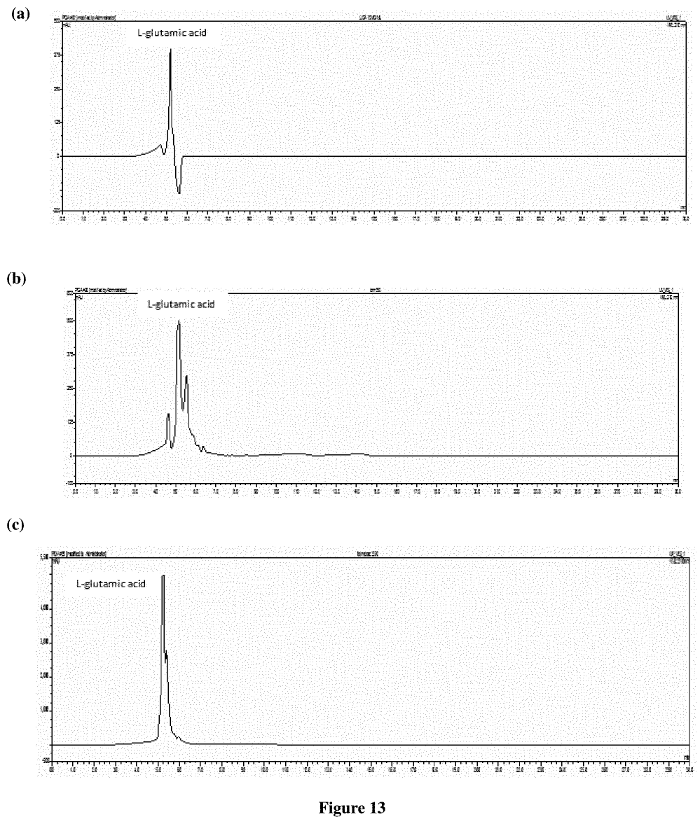

FIG. 15 depicts the increase in γ-PGA productivity using sucrose as the carbon source;

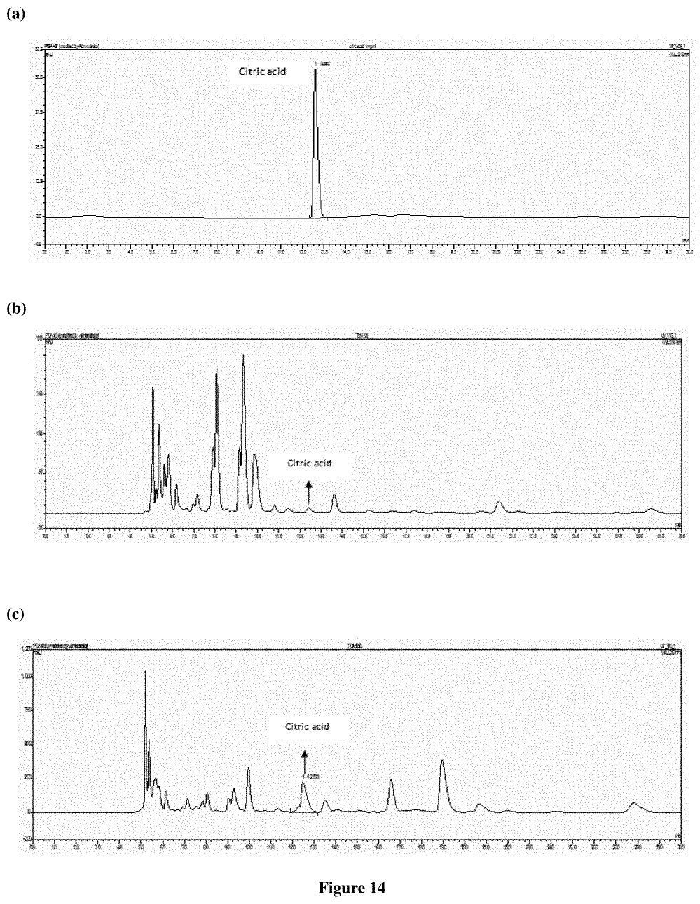

FIG. 16 depicts the non-sterile fermentation under osmophilic condition induced by sucrose.

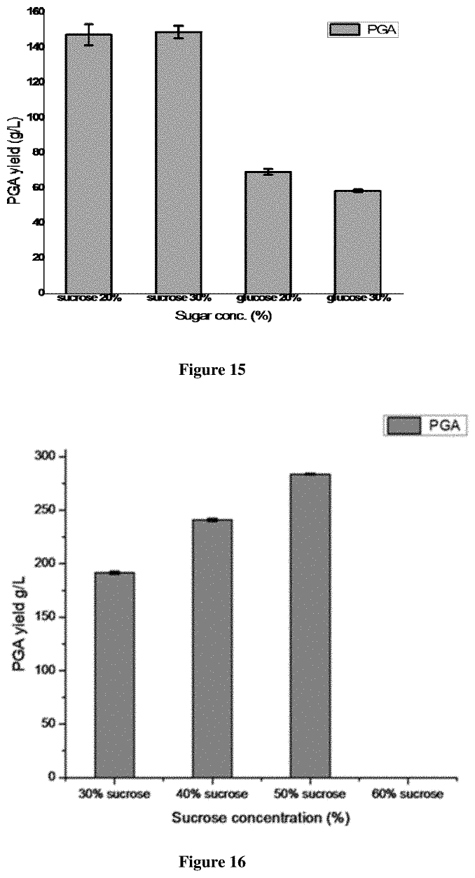

### DETAILED DESCRIPTION OF THE INVENTION

The invention will now be described in detail in connection with certain preferred and optional embodiments, so that various aspects thereof may be more fully understood and appreciated.

Source of Biological Material: Bacillus paralicheniformis H6 MCC 0196.

Tomato waste: Waste of Solanum lycopersicum was obtained from the local market in Pune (India). Sucrose: purchased from local shop in Pune (India).

Tomato waste means over-ripened or lesion containing tomatoes that are usually thrown off as un-suitable for human consumption.

Tomato has all the major nutritional components such as glutamic acid, citric acid, glucose and fructose making it a suitable candidate for the economical production of poly-gamma-glutamic acid (γ-PGA). The present invention provides the potential of tomato waste as substrate to serve as a complete medium by substituting conventional production medium for poly-gamma-glutamic acid synthesis, thereby providing an economically efficient process for the synthesis of γ-PGA.

In an aspect, the present invention provides a process for the synthesis of poly-gamma glutamic acid, comprising cultivating Bacillus paralicheniformis H6 (MCC 0196) in a culture medium comprising tomato waste for 48 h at 28° C. and recovering from the culture medium poly-gamma-glutamic acid released by Bacillus paralicheniformis H6.

Accordingly, the novel γ-PGA producing strain Bacillus paralicheniformis H is isolated from honey which can valorize tomato waste to γ-PGA. Specifically, in the present invention the tomato waste is characterized in that the waste comprises at least 50 mg/ml glucose, fructose 50 mg/ml glutamic acid 10 mg/ml and Citric acid 5 mg/ml.

In a further aspect, the present invention provides a process for the synthesis of highly pure poly-gamma-glutamic acid, comprising cultivating Bacillus paralicheniformis H6 (MCC 0196) in a culture medium comprising tomato waste and optional nitrogen sources and nutrient elements for 48 h at 28° C. and recovering from the culture medium poly-gamma-glutamic acid released by Bacillus paralicheniformis H6 (MCC 0196).

In another aspect, the present invention facilitates reduction in the cost of production associated with organic nitrogen sources in case of synthetic medium. It has also been observed that Bacillus paralicheniformis H6 produces about 40 g/L of γ-PGA in the tomato waste medium in the absence of additional nutrient elements and vitamins, which was higher compared to production in synthetic medium which has been performed in the present invention.

In another aspect, the present invention facilitates increase in γ-PGA production using sucrose as an inexpensive carbon source. The striking feature observed in the optimization studies provides the 2-3 fold elevation in γ-PGA production when the glucose moiety was substituted with sucrose. The maximum γ-PGA yield of 158 g/L was obtained within 48 h using 20% sucrose as the carbon source with the highest productivity of 3.29 g/L/h.

In an advantageous aspect, the present invention provides the ultimate potential of tomato waste and sucrose to completely substitute the synthetic production medium thereby decreasing the overall production cost in the synthesis of γ-PGA. The present invention also entails non-sterile fermentation approach for the highest production and productivity of γ-PGA in the batch fermentation.

In a preferred embodiment, the present invention provides a process for the synthesis of poly-gamma-glutamic acid, comprising cultivating Bacillus paralicheniformis H6 in a culture medium comprising tomato waste and/or high concentrations of sucrose for 48 h at 28° C. and recovering from the culture medium poly-gamma-glutamic acid released by Bacillus paralicheniformis H6.

The present invention provides for isolation of γ-PGA producers from food sources to obtain potentially GRAS (Generally regarded as safe) strains. From all the strains screened for γ-PGA production only one strain namely H6 later designated as Bacillus paralicheniformis H6 showed relatively higher viscosity in the fermentation broth. Therefore, the inventors of the present invention employed Bacillus paralicheniformis H6.

The 16S rRNA gene sequence was searched for closely related species from NCBI database and the H6 isolate was found to be closest relative of Bacillus paralicheniformis KJ-16T with 99% similarity. The 16S rRNA gene sequence is deposited in Genbank (National Centre for Biotechnology Information; NCBI) under the accession number MT138545. The isolate is deposited in IDA collection under accession number MCC 0196).

In a preferred optional embodiment, the present invention provides a process for the synthesis of poly-gamma-glutamic acid, comprising cultivating Bacillus paralicheniformis H6 in a culture medium comprising tomato waste and nutrient elements and sugars for 48 h at 28° C. and recovering from the culture medium poly-gamma-glutamic acid released by the Bacillus paralicheniformis H6.

The present process for synthesis of poly-gamma-glutamic acid production employs using a natural medium consisting of only tomato waste material. The substrate comprising tomato waste material was obtained from over-ripened/lesioned/waste tomatoes which is grounded and centrifuged at 8000 rpm for 20 min. The supernatant obtained, i.e. the debris free tomato juice was collected and was concentrated up to 5× using a rota vapour comprising 50 mL (i.e. 250 mL of supernatant was concentrated to reach a final volume of 50 mL). The pH of the medium was adjusted to 7.5 using 10N NaOH and was sterilized. Accordingly, such a substrate consisting of tomato waste is used for synthesis of poly-gamma-glutamic acid.

In another preferred embodiment, the present invention provides a culture medium comprising tomato waste in a concentration ranging from 4× to 6× by weight of the culture medium, a nitrogen source in a concentration ranging from 0.6% to 1% by weight of the culture medium and nutrient elements in a concentration ranging from 0.01% to 0.1% by weight of the culture medium.

In an embodiment, the present invention provides a process for synthesis of poly-gamma-glutamic acid, wherein the pH of the culture medium was maintained in a range of 4.5 to 7.5 to obtain a significant increase in γ-PGA production.

The sources of carbon, nitrogen, glutamic acid and citric acid in the process may be natural or synthetic. The carbon sources are sugars selected from but not limited to glucose, fructose, sucrose, trehalose, xylose and lactose, In a preferred embodiment, the source of nitrogen are organic sources selected from peptone, tryptone, yeast extract and beef extract or inorganic sources selected from ammonium sulfate, ammonium chloride, ammonium citrate, ammonium acetate and ammonium nitrate. The process further includes a source of potassium, magnesium, calcium and iron.

The molecular weight of the poly-gamma-glutamic acid is in the range of 500 kDa.-1000 kDa. Further, in one preferred embodiment, the present invention provides yield of γ-PGA in a concentration ranging from 20 g/L to 50 g/L within a duration of 2 days.

In one embodiment, the present invention provides tomato waste as a substitute for glucose, L-glutamic acid and citric acid which are the major nutrients for γ-PGA production. The Bacillus paralicheniformis H6, produces about 39 g/L of γ-PGA within 48 h of fermentation at 28° C. using tomato waste alone as the complete medium.

Further, the present invention provides comparison between the production of γ-PGA using a conventionally used optimized synthetic medium and a natural medium to obtain a final PGA yield of 25 g/L. However, in case of natural medium (i.e. tomato waste), no optimization was done other than concentrating the tomato waste liquid to 5× helped achieved a yield of 40 g/L of PGA which is almost twice as compared to synthetic medium.

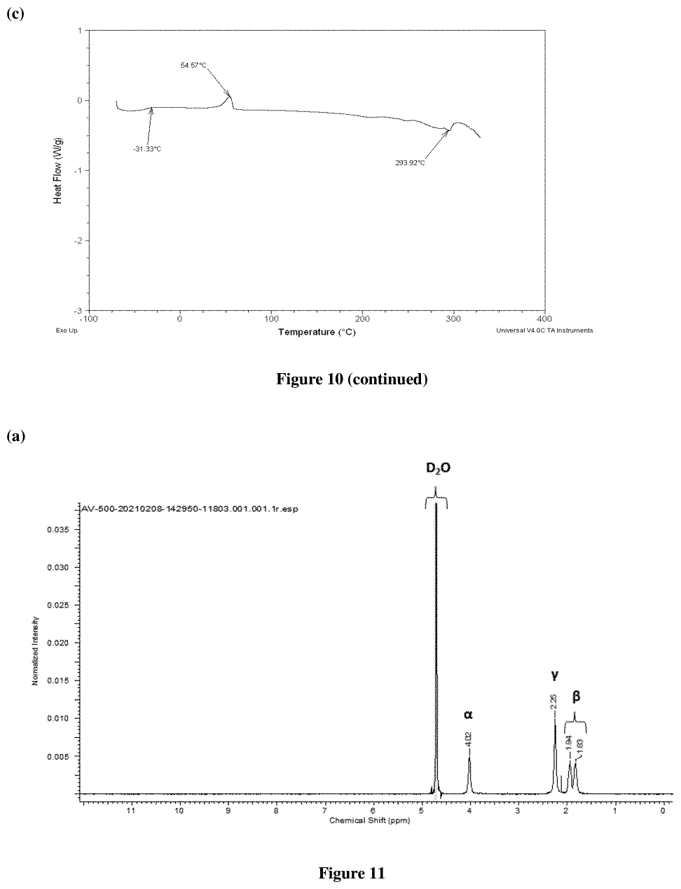

The present invention provides that the breaking point of the poly-gamma-glutamic acid produced by the present process was observed at 300° C. which concludes that the higher thermal stability of the biopolymer as shown in FIG. 10. Additionally, the FTIR spectrum at 3300 cm⁻¹, 1750 cm⁻¹, 1590 cm⁻¹, 720 cm⁻¹ confirms the presence of O—H stretch, C═O stretch, N—H bend and C—H. The peaks observed in the FTIR spectrum confirmed the presence of hydroxyl bond (OH), carbonyl stretch, amide bond.

In another embodiment, as the concentration of glucose is increased from 1% to 30%, the γ-PGA yield significantly increased from 2 g/L to 58 g/L within 48 h. Further the γ-PGA production from glucose is increased to 73 g/L by optimizing the concentration of L-glutamic acid, ammonium nitrate and citric acid. It was observed that a result of 2-3 fold elevation in γ-PGA production when the glucose moiety was substituted with sucrose. The maximum γ-PGA yield of 158 g/L was obtained within 48 h using 20% sucrose as the carbon source with the highest productivity of 3.29 g/L/h. Similarly, maximum γ-PGA yield of 198 g/L was obtained within 48 h using 30% sucrose as the carbon source with the highest productivity of 4.1 g/L/h. Further, the non-sterile fermentation strategy yielded maximum of 284 g/L of PGA using 50% of sucrose with the productivity of 3.94 g/L/h.

In yet another preferred embodiment, the present invention provides a composition comprising poly-gamma-glutamic acid synthesized by Bacillus paralicheniformis H6, in accordance with the process of the present invention.

The present invention provides employing Bacillus paralicheniformis H6 in a composition in the form of vegetative spores and/or cells. The vegetative form of Bacillus paralicheniformis H6 as used in the present invention is in the range of 1% to 5% of the inoculum of the bacteria cultivated in culture medium.

Accordingly, amino acid analysis performed for the biopolymers purified from the present process showed that the resultant biopolymer may contain only glutamic acid monomers which are comparable to standard glutamic acid.

### EXAMPLES

Following examples are given by way of illustration therefore should not be construed to limit the scope of the invention.

### Example 1: Isolation of γ-PGA Producing Strains

Bacteria were isolated from locally available honey (Pune, India) by serially diluting the sample and spread plated onto nutrient agar. The plates were incubated at 37° C. for 24 h. The isolates were maintained in medium consisting of (g/L): Peptone 5 g/L; Sodium chloride 5 g/L; Beef extract 1.5 g/L; Yeast extract 1.5 g/L; Agar 15 g/L.

### Example 2: Screening for γ-PGA Producers

The isolates were grown in synthetic (conventional) production medium containing (g/1); glucose 40 g/1; citric acid 10 g/1; L-glutamic acid 20 g/1; NH₄Cl 6 g/1; K₂HPO₄ 1 g/1; MgSO₄·7H₂O 0.5 g/1; CaCl₂·2H₂O 0.2 g/1; FeCl₃·7H₂O 0.03 g/1; at pH-7.5 and incubated at 28° C. with 180 rpm for 24 h. Based on the viscosity of fermented broth, strains were selected for further studies. The broth was centrifuged at 10000 rpm for 30 min to obtain supernatant to which four volumes of ice-cold methanol was added to get fibrous precipitate presumably γ-PGA. The precipitate was washed thrice with methanol to remove residual components and finally was subjected to lyophilization (Bhunia, 2012, Journal of Biochemical Technology, 3(4)). Out of all the isolates that were screened for γ-PGA production only one isolate namely H6 later designated as Bacillus paralicheniformis H6 showed relatively higher viscosity in synthetic production medium after incubation for 24 h. The supernatant was centrifuged to remove insoluble materials at 10000 rpm for 30 min followed by addition of ice cold methanol to precipitate biopolymer (presumably γ-PGA).

### Example 3: Molecular Identification of γ-PGA Producing Strains

DNA isolation was carried out by CTAB method (Coleman lab, 2017). The Polymerase Chain Reaction (PCR) was carried out using 530F and 800R primers in a final reaction mixture of 50 μl, which consisted of template DNA: 1 μl, PCR buffer: 5 μl, dNTPs: 5 μl, Forward primer (530F) and Reverse primer (800R): 2.5 μl each, Taq polymerase: 0.5 μl, PCR water: 33.5 μl. The PCR was run under the following cycling conditions: Initial denaturation at 94° C. for 5 min, followed by 34 cycles of 94° C. for 30 sec, annealing at 55° C. for 30 sec, extension at 72° C. for 1:30 min and a final extension at 72° C. for 10 min. Purification of the PCR product was carried out using PEG-NaCl protocol. The DNA sequencing was performed on ABI 3500XL genetic analyzer. The 16S rRNA gene sequence obtained was searched for closely related species from the NCBI database by using the https://blast.ncbi.nlm.nih.gov/Blast.cgi. The evolutionary history was inferred using the Neighbor-Joining method. Evolutionary analyses were conducted in MEGA6 software. The H6 isolate is a Gram-positive organism showing production of extracellular exopolysaccharides in nutrient agar plate after incubation at 37° C. for 24 h. The 16S rRNA gene sequence was searched for closely related species from NCBI database and the H6 isolate was found to be closest relative of Bacillus paralicheniformis KJ-16T with 99% similarity.

### Example 4: Production of γ-PGA by Bacillus paralicheniformis H6 Isolate Using Synthetic Medium

A loopful of bacterial cells of Bacillus paralicheniformis H6 were inoculated in 50 mL of nutrient broth and incubated for 24 hrs at 28° C. The cells were diluted such that the final OD₆₀₀ was (OD=1.0, OD=2.0, OD=3.0) with OD=1.0 having concentration of 6×10⁸ CFU [i.e. 6×10⁸ CFU/ml]. The synthetic production medium was inoculated with 1%, 2%, 3% inoculum and incubated at 28° C. with 180 rpm for 24 h. All experiments were carried out in triplicates. At inoculum of 1% (OD₆₀₀ 1.0) yield of about 13 g/L of γ-PGA was observed within 24 h of incubation. Even when the inoculum was increased, there was no significant increase in production of γ-PGA.

### Example 5: Effect of Time on the Production of γ-PGA by Bacillus paralicheniformis H6 Isolate

1% inoculum of OD₆₀₀ 1.0 was added to synthetic production medium and incubated at 28° C. for the following time (12 h, 24 h, 36 h, 48 h, 60 h, 72 h). The resulting fermentation broth was centrifuged at 10000 rpm for 30 min to which ice-cold methanol was added and the biopolymer was lyophilized. The γ-PGA production of 20 g/L was achieved when incubation time was increased to 36 h. However, further increasing the incubation period resulted in decrease of γ-PGA production.

### Example 6: Effect of Temperature on the Production of γ-PGA by Bacillus paralicheniformis H6 Isolate

Inoculum of 1% (OD₆₀₀=1.0) was added to synthetic production medium (pH 7.5) and incubated for 36 h at temperature range (28° C., 37° C., 45° C., 50° C.). The fermented broth was centrifuged, and four volumes of ice-cold methanol was added. The biopolymer was lyophilized for 24 hrs. The γ-PGA production was about 20 g/L at temperature of 28° C. Even by increasing the temperature up to 45° C., there was not much decrease in production indicating that H6 isolate can produce γ-PGA at broad range of temperatures.

### Example 7: Effect of pH on the Production of γ-PGA by B. paralicheniformis H6 Isolate

Inoculum of 1% was added to synthetic production medium with pH range (4.5 to 10) and incubated at 28° C. for 36 h under shaking condition. The pH of the medium was adjusted using 10N NaOH. The supernatant was centrifuged to remove insoluble materials at 10000 rpm for 30 min followed by addition of ice-cold methanol to precipitate biopolymer. At pH 4.5 there was no production of γ-PGA but as the pH was increased to 7.5 there was significant increase in production. The optimum pH was 7.5 with yield of 20 g/L. Further on increasing the pH beyond 7.5, there was decrease in the production of γ-PGA.

### Example 8: Effect of Different Carbon Sources on γ-PGA Production by H6 Isolate

Different carbon sources such as glucose, fructose, sucrose, trehalose, lactose, xylose, etc. were added into the production medium with the concentration of 40 g/L to investigate γ-PGA production. The pH of the medium was adjusted to 7.5 using 10N NaOH and 1% inoculum of OD₆₀₀1.0 was added to this medium followed by incubation at 28° C. for 36 hrs under shaking conditions. With different carbon sources tested for γ-PGA production, it was observed that glucose and fructose where the suitable source of carbon with yield of 20 g/L and 22 g/L.

### Example 9: Effect of Different Nitrogen Sources on γ-PGA Production by Bacillus paralicheniformis H6 Isolate

Four organic nitrogen sources (peptone, tryptone, yeast extract, beef extract,) and five inorganic nitrogen sources (ammonium sulfate, ammonium chloride, ammonium citrate, ammonium acetate, and ammonium nitrate) with concentration of 6.0 g/L were used to investigate γ-PGA production after 36 hrs. Inoculum of 1% was added to this production medium and incubated at 28° C. for 36 h under shaking condition. It was observed that ammonium nitrate showed maximum γ-PGA production of 25 g/L. A striking feature was observed wherein this strain could utilize inorganic nitrogen sources more efficiently than organic nitrogen sources.

### Example 10: 7-PGA Production from the Tomato Waste by B. paralicheniformis H6 Isolate

Tomato waste was used as a natural medium for substituting glucose, L-glutamic acid and citric acid for γ-PGA production and accordingly, the efficacy of tomato waste was checked for its ability to substitute glucose, L-glutamic acid and citric acid. Tomato was ground and the supernatant was collected after centrifugation at 8000 rpm for 20 min. The supernatant was concentrated using rotavapor and ammonium nitrate 0.6%; K₂HPO₄ 1%; MgSO₄ 0.5%; CaCl₂) 0.2%; FeCl₃ 0.03% was added. 1% inoculum of OD600 1.0 was added to this concentrated solution and incubated for 48 h at 28° C. under shaking conditions. The supernatant was concentrated to which ammonium nitrate and trace elements were added. It was observed that as tomato was concentrated from 1× to 5× there was significant increase in γ-PGA production with maximum yield of 40 g/L when tomato was concentrated 5×. Further increasing the concentration to 6× did not increase the γ-PGA production.

### Example 11: Comparative Study of γ-PGA Production from the Synthetic and Tomato Waste Medium by H6 Isolate

To check the efficacy of tomato waste medium to completely replace synthetic medium, tomato waste was used directly without the addition of ammonium nitrate and trace elements. As a control for synthetic medium, conventional medium was used without the addition of ammonium nitrate and trace elements. The pH of the medium was adjusted to 7.5 using 10N NaOH and 1% inoculum of OD₆₀₀1.0 was added to this medium followed by incubation at 28° C. for 36 hrs under shaking conditions. It was observed that synthetic medium with ammonium nitrate and trace elements yielded around 25 g/L of γ-PGA. However, when the medium was devoid of ammonium nitrate and trace elements there was no production indicating the importance of ammonium nitrate and trace elements in γ-PGA production. Similarly, tomato waste medium with ammonium nitrate and trace elements yielded 40 g/L of γ-PGA. When the tomato waste medium was devoid of ammonium nitrate and trace elements the γ-PGA production was 39 g/L (no significant reduction in γ-PGA production). This suggest that tomato waste can serve as a complete medium and totally replace synthetic medium for economic production of γ-PGA.

### Example 12: Scale Up of γ-PGA Production in 1 Liter Fermenter Using Tomato Waste

Tomato waste was grinded, and the supernatant was collected after centrifugation at 8000 rpm for 20 min. 5 L of the supernatant was evaporated to obtain a final volume of 1 L. The pH of the medium was adjusted to 7.5 with 10N NaOH. 5% of the inoculum was added to the production medium in the fermenter. The initial agitation and aeration were maintained at 250 rpm and 1 vvm respectively. The γ-PGA was extracted after 48 h of fermentation at 28° C. with four volumes of methanol. The γ-PGA production started within 24 h of incubation with maximum yield of 40 g/L within 48 h.

### Example 13: Purification of Biopolymer by Dialysis

5% γ-PGA solution was prepared using deionized water and centrifuged at 10000 rpm for 1 h to remove any insoluble materials. The supernatant was desalted by dialysis (MW cut-off 14 kD) for 3 days followed by lyophilization to get pure material (Goto, A., & Kunioka, 1992).

### Example 14: Characterization of γ-PGA

(i) Detection of Glutamic Acid Monomers by Thin Layer Chromatography (TLC):
Amino acid analysis was done for standard γ-PGA and also for biopolymers purified from synthetic as well as tomato waste medium using TLC. 10 mg of partially purified biopolymer was hydrolyzed with 2 ml of 6N HCl at 105° C. for 4 h in a glass vial. Any residual HCl was removed by evaporation in rotavapor followed by its dissolution in 1 mL of deionized water. Amino acid was analysed by TLC using butanol-acetic acid-water (12:5:3) as the solvent system. The TLC was developed by spraying 20 ml of 0.2% ninhydrin in acetone followed by drying (Kambourova et al, 2001). All the 3 biopolymers showed Rf value identical to standard glutamic acid indicating that the biopolymer may contain only glutamic acid monomers.

(ii) Fourier Transform Infrared Spectroscopy (FTIR):
The functional characteristics of polymers were recorded with a Perkin Elmer spectrometer I, FTIR diffused reflectance (DRIFT) mode, USA. The wave numbers (v) of recorded IR-signals were quoted in cm⁻¹ ranging from 4500 to 500 cm⁻¹ with a resolution of 4 cm⁻¹. Each spectrum composed of an average of 8 scans. The FTIR spectrum at 3300 cm−1, 1750 cm−1, 1590 cm−1, 720 cm−1 confirmed the presence of O—H stretch, C═O stretch, N—H bend and C—H. The peaks observed in the FTIR spectrum confirmed the presence of hydroxyl bond (OH), carbonyl stretch, amide bond.

(iii) Differential Scanning Calorimetry (DSC):
The thermal stability of polymer was monitored using Differential scanning calorimetry (DSC). The glass transition temperature [T_{g}] and melting temperature [Tₘ] of the polymers were determined by DSC (Model Q10 DSC, TA Instrument, USA), the temperature ranging from −70 to 330° C. About 5 to 6 mg of the sample (biopolymer) was loaded in a DSC pan, and the pan was sealed by applying pressure. In the first cycle, the sample was equilibrated to −70° C. for 2 min and later heated to 100° C. at 10° C./min. In the second cycle, the sample was quenched to −70° C. at 10° C./min. In the third cycle, the sample was heated to 330° C. at 10° C./min. The same method was followed for all the samples under nitrogen atmosphere at a flushing rate 50 mL/min. With DSC the polymer breaking point was observed at 300° C. which indicates the higher thermal stability of the polymer.

(iv) Nuclear Magnetic Resonance (NMR):
The purity of the γ-PGA produced from synthetic and tomato waste media were determined by ¹H NMR with reference to STD γ-PGA (Sigma Aldrich). 10 mg/ml sample were dissolved in D₂O (deuterium oxide) and ¹H NMR were recorded on Bruker AV 500 MHz. The NMR spectrum showed chemical shift at 4.07 ppm, 2.27 ppm, 1.99 and 1.85 ppm representing α-hydrogen, γ-hydrogen and β-hydrogen atom respectively. Further the NMR spectrum of γ-PGA produced from synthetic medium showed impurities (x). However, the γ-PGA produced from tomato waste did not show any additional peaks indicating its purity similar to STD γ-PGA.

### Example 15: Detection of Sugars, Citric Acid and L-Glutamic Acid from Tomato Waste by HPLC

Tomato waste was grinded, and the supernatant was collected after centrifugation at 5000 rpm for 10 min. The supernatant was concentrated 5× using rota vapor and then passed through 0.2-micron filter prior to HPLC analysis. Standard glucose (50 mg/ml), L-glutamic acid (10 mg/ml), fructose (50 mg/ml) and citric acid (5 mg/ml) is used as control. The detection of sugars was carried out using YMC-Poly amine II column/5 μm; Column size: 250×4.6 mm; Mobile Phase: 75% Acetonitrile; Detector: RI; Flow rate: 1 mL/min. The detection of L-glutamic acid was carried out using ChromeCore C18 column/5 μm; Column size: 250×4.6 mm; Mobile Phase: Methanol: water: formic acid (70:30:0.02); Detector: UV; Flow rate:0.5 mL/min. Finally, the organic acid was investigated by ChromeCore C18 column/5 μm; Column size: 250×4.6 mm; Mobile Phase: 0.1% orthophosphoric acid; Detector: UV; Flow rate: 0.5 mL/min. The HPLC analysis of tomato waste medium (5× concentrated) showed the presence of high level of sugars such as fructose and glucose. Further an increased level of L-glutamic acid was also detected, which is a crucial component for γ-PGA biosynthesis. Finally, the organic acid profiling showed the presence of diverse organic acids including citric acid. This confirms the potential of tomato waste to serve as a complete and cost-effective medium for γ-PGA biosynthesis.

### Example 16: Increasing the γ-PGA Productivity Using Sucrose as the Carbon Source

In the fermentation medium, 20% and 30% glucose was substituted with 20% and 30% sucrose to check its effect on γ-PGA production. Inoculum of 1% was added to the fermentation medium and incubated at 28° C. for 48 h under shaking condition. As 20% and 30% glucose was substituted with sucrose 20% and 30% the γ-PGA production elevated 2-3 folds. With 20% and 30% glucose the maximum yield obtained was 73 g/L and 58 g/L respectively. However, with 20% and 30% sucrose, the maximum γ-PGA yield of 147 g/L and 191 g/L respectively was obtained.

### Example 17: Scale Up of γ-PGA Production in 1 Liter Fermenter

The final optimized medium consisted of Sucrose 200 g/L or 300 g/L; L-glutamic acid 70 g/L; Citric acid 10 g/L; Ammonium nitrate 15 g/L; K₂HPO₄ 1 g/L; MgSO₄·7H₂O 0.5 g/L; CaCl₂·2H₂O 0.2 g/L; FeCl₃·7H₂O 0.03 g/L; at pH-7.5. 1% of the inoculum was added to the fermentation medium in the fermenter. The initial agitation and aeration was maintained at 250 rpm and 1 vvm respectively. The γ-PGA was extracted after 48 h of fermentation at 28° C. with four volumes of methanol. The γ-PGA production started within 24 h of incubation with maximum yield of 158 g/L (in 20% sucrose) and 198 g/L (in 30% sucrose) within 48 h. The highest productivity of 3.29 g/L/h (in 20% sucrose) and 4.1 g/L/h (in 30% sucrose) was obtained using this optimized fermentation medium.

### Example 18: Non-Sterile Fermentation for Poly Gamma Glutamic Acid Production Under Osmophilic Condition Induced by Sucrose

The ability of H6 isolate to produce PGA under high sugar concentration serve as the basis of non-sterile fermentation. The production medium consisted of: Sucrose 300 g/L to 600 g/L; L-glutamic acid 70 g/L; Citric acid 10 g/L; Ammonium nitrate 15 g/L; K₂HPO₄ 1 g/L; MgSO₄·7H₂O 0.5 g/L; CaCl₂·2H₂O 0.2 g/L; FeCl₃·7H₂O 0.03 g/L; at pH-7.5. This medium was used without autoclaving with an inoculum of 10%, followed by incubation for 72 h. The γ-PGA was extracted after 72 h of fermentation at 28° C. with four volumes of methanol. Further, the fermented broth was streaked on nutrient agar plate and incubated at 37° C. for 24 h to check the growth of contaminating microbes in this non-sterile fermentation approach. The γ-PGA production started within 24 h of incubation with maximum yield of 192 g/L (in 30% sucrose), 241 g/L (in 40% sucrose) and 284 g/L (in 50% sucrose) within 72 h. This is the highest γ-PGA production obtained till date in batch fermentation process. However, no PGA production was observed at 60% of sucrose. Further, pure colonies of H6 isolate grew on the nutrient agar plate streaked with the fermented broth.

TABLE 1
A comparison of the reported processes with tomato waste

| | | | | Time | γ-PGA | |
|---|---|---|---|---|---|---|
| Sr. No | Strain | Waste utilized | Additional nutrients | (h) | yield (g/L) | Reference |
| 1 | Bacillus subtilis A3 | Fish meal | Glucose, glutamate | 48 | 25 | Zhang et al., |
| | | wastewater | | | | 2019 |
| 2 | Bacillus licheniformis 9945a | Goose feather | L-glutamate, trisodium citrate, glycerol, | 48 | 5.4 | Altun et al., |
| | | hydrolysate | K₂HPO₄, MgSO₄•7H₂O, FeCl₃, MnSO₄•H₂O, | | | 2019 |
| | | | CaCl₂•2H₂O, ZnSO₄•7H₂O, MnCl₂•4H₂O, | | | |
| | | | H₃BO₃, CoCl₂•6H₂O, CuCl₂•2H₂O, | | | |
| | | | NiCl₂•6H₂O, Na₂MoO₄•2H₂O | | | |
| 3 | Bacillus licheniformis WX-02 | Paper waste | glutamate, sodiumcitrate trihydrate, sodium | 36 | 6.46 | Scheel et al., |
| | | hydrolysate | nitrate, ammonium chloride, potassium phosphate | | | 2019 |
| | | | trihydrate, MgSO₄•7H₂O, ZnSO₄•7H₂O, | | | |
| | | | CaCl₂•2H₂O, MnSO₄•H₂O, | | | |
| 4 | Bacillus sp. SJ-10 | Macroalgae | Sucrose, NH4Cl, L-Glutamate, NaCl, MgSO₄•7H₂O, | 48 | 6.29 | Kim et al., |
| | | (Ulva) | CaCl₂•2H₂O, FeCl₃, MnSO₄•H₂O | | | 2019 |
| 5 | Bacillus subtilis HB-1 | Corncob hydrolysate | glutamate, yeast extract, NaCl, MgSO4, CaCl2 | 40 | 24.29 | Zhu et al., |
| | | | | | | 2014 |
| 6 | Bacillus paralicheniformis H6 | Tomato waste | — | 48 | 40 | This study |

### Advantages of the Invention

Globally around 3 million metric tonnes of tomato wastes is generated. Moreover, India is the 2^{nd} largest tomato producer after China. Therefore, the present invention provides a beneficial process that is best suited for possible utilization of surplus tomato waste to improve economic sustainability and waste valorization.
The present Bacillus paralicheniformis H6 strain has not been reported earlier for γ-PGA production.
Tomato waste can be used as a substitute for glucose, L-glutamic acid and citric acid which are the major nutrients for γ-PGA production. This strain produces about 40 g/L of γ-PGA within 48 h of fermentation at 28° C. using tomato waste alone as the complete medium.
Effective valorization of tomato waste into a value added γ-PGA.
The present process will facilitate the commercialization of γ-PGA by leaps and bounds.
The present invention employs only tomato waste as the substrate required to produce highly pure γ-PGA, therefore, γ-PGA shows 99% decrement of cost compared to conventional process that employ expensive media components.
The present invention also employs non sterile fermentation approach under sucrose mediated osmophilic condition for the highest production and productivity of γ-PGA thereby reducing the cost associated with sterilization.

1. A process for the synthesis of poly-gamma-glutamic acid, wherein, the process comprises steps of:
incubating a raw material having ingredients selected from the group consisting of at least 20% w/w of a carbon, at least 7% w/w of a glutamic acid, at least 1% w/w of a nitrogen, at least 1% w/w of a citric acid and 6×10⁸ to 6×10⁹ CFU/ml of Bacillus paralicheniformis MCC 0196 for 12-48 hours at 28-45° C. at 5.0-8.0 pH to obtain a broth with a supernatant; and
separating the broth and the supernatant, adding ice cold methanol to the supernatant to obtain the poly-gamma-glutamic acid of molecular weight in the range of 500-1000 kDa and a yield ranging from 40-300 g/lt.

2. The process as claimed in claim 1, wherein the Bacillus paralicheniformis MCC 0196 is isolated from honey.

3. The process as claimed in claim 1, wherein the carbon, nitrogen, glutamic acid and citric acid are from a natural source or a synthetic source.

4. The process as claimed in claim 3, wherein the carbon is selected from the group consisting of glucose, fructose, sucrose, trehalose, xylose and lactose and wherein the source of nitrogen are organic sources selected from the group consisting of peptone, tryptone, yeast extract and beef extract or inorganic sources selected from the group consisting of ammonium sulfate, ammonium chloride, ammonium citrate, ammonium acetate and ammonium nitrate.

5. The process as claimed in claim 1, wherein the raw material further comprises ingredients selected from the group consisting of a source of potassium, magnesium, calcium and iron.

6. The process as claimed in claim 3, wherein the natural source of carbon, nitrogen, glutamic acid and citric acid is a tomato waste.

7. The process as claimed in claim 6, wherein said tomato waste is used alone or in combination with the source of potassium, magnesium, calcium and iron.

8. The process for the synthesis of poly-gamma-glutamic acid as claimed claim 1 further comprises:
a) incubating a raw material having ingredients selected from the group consisting of at least 20% w/w of a carbon source, at least 7% w/w of glutamic acid, at least 1% w/w of a nitrogen source, at least 1% w/w of citric acid, NH₄Cl 6 g/lt; K₂HPO₄ 1 g/lt; MgSO₄·7H₂O 0.5 g/lt; CaCl₂·2H₂O 0.2 g/lt; FeCl₃·7H₂O 0.03 g/lt and 6×10⁸ CFU/ml of Bacillus paralicheniformis MCC 0196 for 36 hours at 28° C. at 7.5 pH to obtain a broth with a supernatant; and
b) centrifuging the broth at 10000 rpm for 30 minutes to obtain a clear supernatant and adding ice cold methanol to precipitate the poly-gamma-glutamic acid.

9. A culture medium for the synthesis of poly-gamma-glutamic acid, wherein, the culture medium comprising a tomato waste having at least 50 mg/ml glucose, 50 mg/l fructose, 10 mg/ml glutamic acid and 5 mg/ml citric acid.

10. The culture medium as claimed in claim 9, wherein the culture medium is prepared by a process comprising:
a) grinding the tomato waste;
b) centrifuging the ground tomato waste to obtain a debris free supernatant; and
c) concentrating the supernatant and adjusting the pH to 7.5 to obtain said culture medium.
